(19) United States
(12) Patent Application Publication
(10) Pub. No.: US 2010/0166888 A1
(43) Pub. Date: Jul. 1, 2010

(54) Composition and method for the control of organoleptic properties of bodily fluids and secretions

(76) Inventors: Howard Luber, Paradise Valley, AZ (US); Eugene Gans, Westport, CT (US)

Correspondence Address: REED SMITH LLP, 2500 ONE LIBERTY PLACE, 1650 MARKET STREET, PHILADELPHIA, PA, 19103, US

(21) Appl. No.: 12/319,130

(22) Filed: Dec. 31, 2008

(51) Int. Cl. A61K 33/00; A61K 31/409; A61K 36/00; A61K 31/555

(52) U.S. Cl. 424/717; 514/410; 424/777; 514/185

(57) ABSTRACT

Methods and edible compositions that improve the organoleptic properties of bodily fluids and secretions are provided. The compositions may contain chlorophyll and/or charcoal based compounds which neutralize in whole or in part undesirable organoleptic properties of bodily secretions, and which may also contain compounds which can impart desirable organoleptic properties to the body and to bodily fluids and secretions. The compositions according to this invention may also include compounds which: (1) promote the effective and prompt absorption by the gastrointestinal tract and target fluids of the compounds that can affect the organoleptic properties of the body and of bodily fluids and secretions, and/or (2) enhance the actions of these compounds on the organoleptic properties of the body and of bodily fluids and secretions once the compounds are absorbed by the gastrointestinal tract.

### FIELD OF THE INVENTION

This invention relates to the control and improvement of organoleptic properties of the body and also of bodily fluids and secretions, specifically their taste and smell.

### BACKGROUND OF THE INVENTION

It is highly desirable to control and, if possible, improve the organoleptic properties of bodily fluids and secretions. At a minimum it is important to reduce and, if possible, eliminate organoleptic properties of one's bodily fluids and secretions that one may find unpleasant, or which may give offense to others. If possible, it is also desirable to affect the organoleptic properties of one's bodily fluids and secretions so that they are pleasing to oneself and to others.

Conventionally, people have used a variety of compositions to affect the organoleptic properties of the body and also of bodily fluids and secretions. Among these are mouthwashes, and douches, as well as deodorant compositions that are sprayed, rubbed or otherwise applied to regions of the body such as the arm pits. All of these techniques operate by altering the organoleptic properties of bodily secretions after they have been secreted. They do not affect inherent organoleptic properties of these substances as they are secreted. Also, these methods are often cumbersome and inconvenient. Repeated use of mouthwashes, douches, or application of deodorants during the course of a day can be impossible as a practical matter, even though this sometimes may be needed for effective control of organoleptic properties of bodily secretions. These methods also have unwanted and undesirable side effects in some cases. These side effects can include the development of skin rashes, a deleterious effect on the microflora (including the development of yeast infections), and irritation of mucus membranes. Moreover, these techniques are effective only where locally applied, and have no usefulness with respect to many bodily fluids and secretions that are not available, or poorly available, to local treatment.

It has also been known for some time that diet can affect the organoleptic properties of bodily fluids and secretions. Certain foods, such as asparagus and curry, are well known for their effect on the organoleptic properties of certain bodily fluids and secretions.

It is also known that fruits, sweets, and to some extent spices, can when eaten in sufficient quantities, have a favorable effect on the organoleptic properties of bodily secretions. See, for example, U.S. Pat. No. 6,485,773. This technique is not free of serious shortcomings. Its effectiveness is limited by the amount of these materials which one may ingest without consuming an unacceptably large number of calories. When consumed in quantities sufficient to have a positive effect on organoleptic properties, they can also result in the generation of intestinal gas, leading to an uncomfortable sense of bloating and flatulence, which may be offensive in its own right. Another disadvantage of this technology is that it often requires “flushing” of fluids in order to be effective. See, for example, U.S. Pat. No. 6,485,773 which calls for the user to follow such a procedure with respect to semen so that the organoleptic properties of subsequently generated semen can then be improved. After “flushing,” this technology still requires 12-14 hours to work, and it is actually recommended that it be used as long as 2 days for it to reach effectiveness. This requires an often-unacceptable amount of advance planning, or the continuous and systematic use of the product. These are serious disadvantages that limit the practical application of the prior art technology. Also, the consumption of significant quantities of sugars or sugary fruits can be completely unacceptable for persons having special dietary needs, such as diabetics and those on calorie restricted diets.

Therefore, there exists a need for an improved, more practical, method of regulating and, if possible, improving the organoleptic properties of the body and also of bodily fluids and secretions.

### SUMMARY OF THE INVENTION

According to this invention, there are provided edible compositions that may contain chlorophyll and/or charcoal based compounds which neutralize in whole or in part undesirable organoleptic properties of bodily secretions, and which may also contain compounds which can impart desirable organoleptic properties to the body and to bodily fluids and secretions. Edible compositions according to this invention may also include compounds which: (1) promote the effective and prompt absorption by the gastrointestinal tract and target fluids of the compounds that can affect the organoleptic properties of the body and of bodily fluids and secretions, and/or (2) enhance the actions of these compounds on the organoleptic properties of the body and of bodily fluids and secretions once the compounds are absorbed by the gastrointestinal tract.

This invention also provides methods for using the above-described compositions to improve the organoleptic properties of the body and of bodily fluids and secretions.

### DETAILED DESCRIPTION OF THE INVENTION

Edible compositions according to this invention can contain chlorophyll and/or charcoal based compounds which neutralize undesirable organoleptic properties of bodily secretions, compounds which impart desirable organoleptic properties to bodily secretions, and compounds which promote the absorption and/or action of compounds which affect the organoleptic properties of bodily secretions. Edible compositions according to this invention are most conveniently provided in the form of tablets or gel caps. They may also be provided in the form of powders which can be added to foods or dissolved in water to form a drink or made as lozenges, gels or bars. Whichever form is used, the edible compositions according to this invention may also include conventional adjuvants and carriers such as tabletting aids, anti-caking agents, colorings, encapsulants, emulsifiers, and the like.

According to the invention, the edible compositions of this invention can include chlorophyll and/or charcoal based compounds which negate in whole or in part undesirable organoleptic properties of bodily fluids and secretions. These compounds include: chlorophyll, charcoal, chlorophyll derivatives such as copper chlorophyllin, thymol, eucalyptol and other aromatic compounds, menthol and other mints, bismuth compounds, tea tree oil and other antimicrobial compounds, silicates and other absorbants and absorbants and agents that can bind, complex, or otherwise inactivate malodorous agents.

A variety of compounds which can impart desirable organoleptic properties may be used in the edible compositions according to this invention. Sugars, natural and artificial food flavorings, and food extracts can be used, as can certain spices. These can include vanilla, ginger, pineapple, cinnamon, banana, cranberry, menthol, pomegranate vanillin, fennel, lavender, rosemary oil, bergamot oil, lemon, lime, orange, rose and rose oil, and jasmine. Natural extracts, concentrates and synthetic derivatives can be used. These ingredients may be obtained from a variety of sources. Most are available from commercial suppliers in liquid form, as dry powders, as lyophilized powders, or in other convenient forms.

Edible compositions according to this invention may also include compounds which promote the absorption of the above compounds by the gastrointestinal tract and/or enhance the migration of those compounds to the secretory tissues, and/or the inclusion of those compounds in the secretions. Most preferably, these compounds are lipophilic. Examples of these compounds are food oils. Food oils include compositions such as wheat germ oil, flax oil, safflower oil, parsley seed oil sesame oil, sweet almond oil, citrus oils, sunflower seed oil, meadowfoam oil, corn oil, cottonseed oil, olive oil, peanut and other nut oils, soybean oil, soy lecithin, mineral oil, low molecular glycerides and other natural and synthetic oils. These are widely available commercially as food and/or medicinal grade quality ingredients.

The use of chlorophyll and/or charcoal based compounds which negate undesirable organoleptic properties can reduce or, in some cases, eliminate the need for compounds which impart desirable organoleptic properties to bodily secretions. This feature of the invention is particularly useful for persons who are on calorie restricted diets, are diabetic, or have food allergies to the other ingredients.

Probiotics, which are known in the art, can also be used in compositions of this invention. Probiotics have the unexpected effect of enhancing the action of these compositions on organoleptic properties, and probiotics can further reduce unwanted gastrointestinal effects.

By using lipophilic compounds, such as food oils, in edible compositions of the present invention, the action of the compounds which negate undesirable organoleptic properties or which impart desirable organoleptic properties is greatly enhanced. This effect exists to such a degree that a significantly smaller quantity of those ingredients will achieve a much stronger effect than is the case when lipophilic compounds are not included. This makes possible the use of lesser quantities of the compositions according to this invention, which enhances the convenience of its use and reduces the incidence and severity of undesirable gastrointestinal effects such as bloating and gas generation, and reduces the caloric load imposed by the use of the composition.

The action of compounds which negate undesirable organoleptic properties and/or compounds which impart desirable organoleptic properties can also be enhanced by including in the edible products of this invention, agents that can improve gastric components. This can be accomplished by compounds capable of neutralizing undesirable gastric components, by compounds capable of enhancing gastric components, by enzymes which may aid absorption, and/or by agents that raise gastric pH to enhance and stimulate the transport the agents of this invention to intestinal absorption sites. Examples are sodium and other bicarbonate and carbonates, and silicates that can absorb/absorb gastric acids and other components. It is believed that these may aid absorption in the gut and/or aid transport of these compounds to the secretory tissues. These can be added separately, but are most conveniently and effectively added by mixing with the chlorophyll and/or charcoal components before the final formulation is combined.

The compositions and methods of this invention can result in an improvement in the organoleptic properties of the body and also of bodily fluids and secretions. In particular, improvement can be effected in the organoleptic properties of sweat, semen, vaginal secretions, and saliva.

The compositions of the present invention will now be illustrated by the following non-limiting examples.

### EXAMPLE 1

A capsule according to the invention can be made using the following ingredients:

| | Per Capsule |
|---|---|
| Pineapple Powder-Freeze Dried | 0.18 g |
| Banana Powder-Freeze Dried | 0.18 g |
| Organic French Vanilla Powder | 0.18 g |
| Cinnamon Powder | 0.06 g |
| Sodium Bicarbonate-Chlorophyll Mixture | 0.05 g |
| Wheat Germ Oil | 0.05 g |
| Flax Oil | 0.10 g |
| Safflower Oil | 0.10 g |
| Sesame Oil | 0.10 g |
| Total: | 1.00 g |

The Sodium Bicarbonate-Chlorophyll Mixture is prepared as follows: Five (5.00) grams of “ChlorOxygen” Chlorophyll Liquid Concentrate or other sources of chlorophyll such as sodium copper chlorophyllin or other various commercially available concentrates of chlorophyll are mixed very well with 75.0 (seventy-five) grams of dry sodium bicarbonate. This damp powder is spread on a tray and dried at 30° C. to 37° C. for 24 to 48 hours.

The green-colored, Chlorophyll-Bicarbonate mixture is then added to the French Vanilla Powder and mixed until the green color is uniformly dispersed. The other powders are then added and mixed thoroughly. Then, in a separate vessel, the oils are mixed. The mixed oils are then slowly added to the powders, mixing and blending thoroughly to create a powder mass. This powder mass can then be filled by hand, or by mechanical devices, into various edible capsules. For instance, two-piece gelatin cellulose or other two-piece capsules, where the capsules are opened and the mass is pressed into the top and/or bottom of the capsule according to the weight of mass desired, and the two parts of the capsule are then pushed together and closed. Alternately, the mass can also be incorporated into single piece gelatin or other polymer capsules, using devices that can extrude the desired amount of the mass into the capsule as it is being formed. In these examples, the amounts of the liquid and solid components of the formula can be adjusted to facilitate filing the mass into the capsules.

### EXAMPLE 2

Another capsule according to the invention is made using the following ingredients:

| | Per Capsule |
|---|---|
| Artificial Pineapple Flavor Powder | 0.20 g |
| Natural/Artificial Banana Flavor Powder | 0.18 g |
| Artificial Vanilla Flavor Powder | 0.18 g |
| Natural Cinnamon Flavor Powder | 0.025 g |
| Natural Ginger Flavor Wonf Powder | 0.025 g |
| Sodium Bicarbonate-Chlorophyll Mixture | 0.06 g |
| Flax Oil | 0.10 g |
| Wheat Germ Oil | 0.03 g |
| Safflower Oil | 0.10 g |
| Sesame Oil | 0.10 g |
| Total: | 1.00 g |

These ingredients are mixed and filled into capsules in the same manner as described in Example 1.

### EXAMPLE 3

Another capsule according to the invention is made using the following ingredients:

| | Per Capsule |
|---|---|
| Wheat Germ Oil | 0.080 g |
| Flax Oil | 0.160 g |
| Safflower Oil | 0.160 g |
| Sesame Oil | 0.160 g |
| Natural Cinnamon Flavor Powder | 0.025 g |
| Artificial Vanilla Flavor Powder | 0.140 g |
| Banana Flavor Powder | 0.140 g |
| Natural Ginger Flavor Powder | 0.025 g |
| Pineapple Flavor Powder | 0.170 g |
| Sodium Bicarbonate | 0.060 g |
| Chlorophyllin Na—Cu (E141) USP | 0.40 mg |
| Mono & Diglycerides of fatty acids and soybean | 1.152 g |
| lecithin qs to a fill weight of | |

These ingredients are mixed and filled into capsules in the same manner as described in Example 1.

The amount of each ingredient used in each dose of the compositions of this invention and the total dose used will vary in light of several factors. These factors include the frequency of dosing, the person's body weight, the strength of the organoleptic properties of that person's secretions, and whether one wishes to impart desirable organoleptic properties or to simply control undesirable organoleptic properties. These factors are idiosyncratic and reflect personal preferences. It is not possible to specify the exact quantities that should be used in any individual to achieve a specific organoleptic result. However, the quantities described in the above examples are generally enough to give a satisfactory result in most cases when administered once or twice. If this is found to be insufficient, the dosing can be easily increased, without deleterious side effects.

With respect to the chlorophyll and/or charcoal based compounds that neutralize undesirable organoleptic properties, these can be administered in the amount of—chlorophyll from the equivalent of 0.05 mg to 5.0 mg of pure chlorophyll, more commonly in the amount from 0.25 to 1.0 mg and for charcoal, from 0.010 g to 1.0 g per dose, more commonly in an amount of from 0.050 g to 0.05 g per dose.

With respect to compounds that impart desirable organoleptic properties, these can be, as a group, administered in an amount of from 0.05 g to 5.0 g per dose, more commonly in a range of from 0.20 g to 2.0 g per dose, in a single or divided doses.

Compounds, such as food oils, which enhance the absorption of the above ingredients can be included with the above ingredients in an amount of from 0.05 g to 5.0 g, more commonly in an amount of from 0.20 g to 2.0 g. Alternatively, they may be included so that the ratio of the weight of these compounds to the weight of the compounds that affect the organoleptic properties of bodily secretions is from 0.2:1 to about 5.0:1, more commonly from 0.5:1 to 2:1. (e.g. 0.5 oil:1 other ingredient).

When provided in a gelcap, tablet, or capsule form, the edible composition of this invention should be formulated so as to readily dissolve or break up in the stomach and/or small intestines, thereby making the ingredients available for absorption. This point is less important when the edible compositions of the invention are in forms such as powders, drinks or chewable tablets.

Edible compositions of this invention were tested and found to have a noticeable positive effect on the organoleptic properties of bodily fluids and secretions. They also have a noticeable positive effect on the general organoleptic properties of the body itself.

The invention, such as in the form of capsules according to the above examples, can be taken with water and is effective within as little as 1 to 2 hours. No “flushing” of existing bodily fluids is required for improvement in organoleptic properties to be obtained. This is a remarkable improvement over prior art compositions, and permits the spontaneous or unplanned use of compositions or methods of the invention when desired or needed.

### Clinical Studies

Double-blinded studies of the effectiveness of this invention were conducted. Participants were enrolled in the study, and their partners reported their evaluations of organoleptic properties. The study protocol, including the enrollment of participants, was reviewed and approved by an independent IRB to ensure compliance with applicable scientific, safety, and ethical standards.

### First Clinical Study

In the first part of the study, a baseline was established. Fourteen participants had their partners note the taste and smell of their semen and vaginal secretions, without the use of any supplements. Two observations were made, no more than 1 day apart. Next, the participants took two capsules and their partners evaluated the organlopetic properties of their secretions anywhere from 1 to 13 hours afterward. This was repeated the next day. Six of the fourteen partners reported an improvement in taste and/or smell of semen and vaginal secretions. This procedure was repeated, with the participants taking two capsules twice a day (i.e. morning and evening). Seven of the fourteen partners reported improvement in the taste and/or smell of semen and vaginal secretions. Next, this procedure was repeated after a period during which the participants did not use the composition of this invention. The partners' evaluations returned to baseline, with some reporting a “chlorine” taste or smell, and none reported a pleasant taste or smell. Finally, this procedure was performed a fifth time, with the participants consuming two capsules two hours before evaluation. This resulted in eight of the fourteen evaluators reporting improved smell and/or taste. None of these procedures included a “flushing” of the fluids.

### Second Clinical Study

In the first part of the study, a baseline was established. Six participants had their partners note the taste and smell of their semen and vaginal secretions. Next, the participants took two capsules formulated according to the invention twice a day and half of their partners reported an improvement in taste and/or smell. This procedure was repeated without using any formulations according to the invention, and the partners' evaluation of taste and smell returned to baseline. Finally, this procedure was repeated, with the participants taking two capsules 3-8 hours before evaluation. Two thirds of the partners reported an improvement in taste and/or smell. None of these procedures included a “flushing” of the fluids.

The invention will now be described in the following claims.

We claim:

1. An edible composition comprising:
a) a compound for negating undesirable organoleptic properties selected from the group consisting of chlorophyll, chlorophyll derivatives, charcoal, and charcoal derivatives, and
b) at least one food oil.

2. The edible composition of claim 1, further comprising agents to improve gastric components.

3. The edible composition of claim 2, wherein the agent to improve gastric components is sodium bicarbonate.

4. The edible composition of claim 2, further comprising agents for the improvement of organoleptic properties.

5. The edible composition of claim 4, wherein the agent for the improvement of organoleptic properties is selected from the group consisting of sugars, lyophylized fruit, fruit extracts, essential oils of fruits, and spices.

6. The edible composition of claim 5, wherein the compound for negating undesirable organoleptic properties is selected from the group consisting of chlorophyll and chlorophyll derivatives.

7. The edible composition of claim 6, wherein the compound for negating undesirable organoleptic properties is chlorophyllin copper complex sodium.

8. The edible composition of claim 4, 5, 6 or 7 wherein at least one food oil is selected from the group consisting of wheat germ oil, flax oil, safflower oil, soybean oil, olive oil, peanut oil, mineral oil, parsley seed oil, sesame oil, diglycerides of fatty acids, monoglycerides of fatty acids, and soy lecithin.

9. The edible composition of claim 4, 5, 6, or 7 wherein the agent for the improvement of organoleptic properties is selected from the group consisting of banana powder, vanilla extract, artificial vanilla powder, pineapple juice, pineapple powder, cinnamon, ginger root, ginger, cranberry, strawberry extract, strawberry powder, cherry juice, cherry extract, and cherry powder.

10. The edible composition of claim 8, wherein the agent for the improvement of organoleptic properties is selected from the group consisting of banana powder, vanilla extract, artificial vanilla powder, pineapple juice, pineapple powder, cinnamon, ginger root, ginger, cranberry, strawberry extract, strawberry powder, cherry juice, cherry extract, and cherry powder.

11. A method for improving the organoleptic properties of a person's bodily fluids or secretions, comprising administering to the person an edible composition comprising:
a) a compound for negating undesirable organoleptic properties selected from the group consisting of chlorophyll, chlorophyll derivatives, charcoal, and charcoal derivatives, and
b) at least one food oil.

12. The method of claim 11, wherein the edible composition further comprises agents to improve gastric components.

13. The method of claim 12, wherein the agent to improve gastric components is sodium bicarbonate.

14. The method of claim 12, wherein the edible composition further comprises agents for the improvement of organoleptic properties.

15. The method of claim 14, wherein the agent for the improvement of organoleptic properties is selected from the group consisting of sugars, lyophylized fruit, fruit extracts, essential oils of fruits, and spices.

16. The method of claim 15, wherein the compound for negating undesirable organoleptic properties is selected from the group consisting of chlorophyll and chlorophyll derivatives.

17. The method of claim 16, wherein the compound for negating undesirable organoleptic properties is chlorophyllin copper complex sodium.

18. The method of claim 11, 12, 13, 14, 15, 16 or 17 wherein at least one food oil is selected from the group consisting of wheat germ oil, flax oil, safflower oil, soybean oil, olive oil, peanut oil, mineral oil, parsley seed oil, sesame oil, diglycerides of fatty acids, monoglycerides of fatty acids, and soy lecithin.

19. The method of claim 14, 15, 16, or 17 wherein the agent for the improvement of organoleptic properties is selected from the group consisting of banana powder, vanilla extract, artificial vanilla powder, pineapple juice, pineapple powder, cinnamon, ginger root, ginger, cranberry, strawberry extract, strawberry powder, cherry juice, cherry extract, and cherry powder.

20. The method of claim 18 wherein the agent for the improvement of organoleptic properties is selected from the group consisting of banana powder, vanilla extract, artificial vanilla powder, pineapple juice, pineapple powder, cinnamon, ginger root, ginger, cranberry, strawberry extract, strawberry powder, cherry juice, cherry extract, and cherry powder.

21. The method of claim 11, 12, 13, 14, 15, 16, or 17 wherein the bodily fluid or secretion is semen.

22. The method of claim 11, 12, 13, 14, 15, 16, or 17 wherein the bodily fluid or secretion is vaginal secretion.

23. The method of claim 19 wherein the bodily fluid or secretion is semen.

24. The method of claim 19 wherein the bodily fluid or secretion is vaginal secretion.

25. The method of claim 11, 12, 13, 14, 15, 16, or 17 wherein the bodily fluid or secretion is sweat.

26. The method of claim 19 wherein the bodily fluid or secretion is sweat.
